(19) United States
(12) Patent Application Publication
(10) Pub. No.: US 2010/0094066 A1
(43) Pub. Date: Apr. 15, 2010

(54) GAS PHASE REACTION PROCESS FOR POLYHYDRIC COMPOUNDS

(75) Inventor: Galen J. Suppes, Columbia, MO (US)

Correspondence Address: POLSINELLI SHUGHART PC, 700 W. 47TH STREET, SUITE 1000, KANSAS CITY, MO, 64112-1802, US

(73) Assignee: THE CURATORS OF THE UNIVERSITY OF MISSOURI, Columbia, MO (US)

(21) Appl. No.: 12/523,363

(22) PCT Filed: Jan. 17, 2008
(86) PCT No.: PCT/US08/51257

§ 371 Date: Dec. 29, 2009

Related U.S. Application Data
(60) Provisional application No. 60/880,870, filed on Jan. 17, 2007.
(60) Provisional application No. 60/962,630, filed on Jul. 31, 2007.

(51) Int. Cl. C07C 31/18

(52) U.S. Cl. 568/861

(57) ABSTRACT

The present invention provides processes for the gas phase conversion of a polyhydric feedstock into an oxygen-containing product. The polyhydric feedstock comprises water and at least one polyhydric compound having from about four to about twelve carbon atoms and more than three hydroxyl groups. Also provided are processes for the separation of the oxygen-containing product from the reaction product mixture.

### FIELD OF THE INVENTION

This invention relates to the gas phase processing of polyhydric compounds having from about four to about twelve carbon atoms into oxygen-containing products.

### BACKGROUND OF THE INVENTION

The processing of sugars having three or more carbon atoms has traditionally been performed in liquid phase. For example, U.S. Pat. Nos. 5,276,181, 5,214,219, 5,616,817, 4,642,394, 2,852,270, 3,030,429, 4,404,411, 5,600,028, 4,366,332, and 4,433,184 provide examples of known methods for converting glycerol (a three carbon sugar) and/or sugars having more than three carbon atoms into products having commercial value. Typically, these reactions are conducted in aqueous (liquid) phases with hydrogen present at pressures above 100 bars. Catalyst deactivation and recovery are generally problematic with liquid phase reactions, however.

Gas phase packed-bed reactor processes have operational advantages over slurry reactions primarily because solid catalysts are easier to remove from gases than from liquids. Additionally, gas phase reactions tend to have greater product recovery and less catalyst deactivation than liquid phase reactions. For these reasons, the processing of sugars in gas phase reactions would be beneficial. Sugars, however, are generally considered non-volatile. Suppes and Sutterlin (WO 2007/053705) recently disclosed that glycerol could be evaporated and maintained in gaseous phase, thereby providing a higher selectivity and an extended catalyst life. U.S. Pat. No. 5,387,720 also revealed a gas phase reaction process, but it was limited to temperatures above 250° C. due to inadequate approaches to overcome the low vapor pressure of glycerol at lower temperatures. Although glycerol can be reacted in gas phase under specific reaction conditions, it is unknown whether sugars having four or more carbon atoms could also be evaporated and reacted in gas phase. One challenge is that the boiling points of sugars having four or more carbon atoms are much higher than the boiling point of glycerol (which is about 290° C.). Thus, there is a need for processes for the gas phase conversion of a sugar having four or more carbon atoms into a commercially valuable product(s). Furthermore, there is a need for separation processes to efficiently recover the reaction products.

### SUMMARY OF THE INVENTION

Among the various aspects of the present invention is one aspect that provides a process for converting a polyhydric feedstock into an oxygen-containing product. The polyhydric feedstock comprises at least about 10% of water by weight and at least about 2% by weight of at least one polyhydric compound having from about four to about twelve carbon atoms and more than three hydroxyl groups. The process comprises evaporating the feedstock at a temperature from about 150° C. to about 340° C. and at a pressure from about 0.01 bars to about 200 bars to form a substantially gas phase reaction mixture. The process further comprises contacting the gas phase reaction mixture with a heterogeneous catalyst to facilitate a reaction such that the oxygen-containing product is formed.

Another aspect of the invention encompasses a process for separating an oxygen-containing product from a reaction product mixture comprising a polyhydric compound and hydrogen. The process comprises introducing the reaction product mixture into a dividing wall column. The dividing wall column comprises a rectifying section comprised of more than four stages, a walled rectifying section comprised of more than four stages, a walled stripping section comprised of more than eight stages, and a stripping section comprised of more than eight stages. The process further comprises collecting the oxygen-containing product.

A further aspect of the invention provides a process for converting a three-carbon alcohol feedstock into acrolein. The process comprises evaporating the alcohol feedstock at a temperature from about 180° C. to about 250° C. and at a pressure from about 0.03 bars to about 5 bars to form a substantially gas phase reaction mixture. The process further comprises contacting the reaction mixture with a heterogeneous catalyst at a temperature from about 200° C. to about 250° C., wherein the catalyst has a Hammett acidity value from about −2 to about −10.

Still another aspect of the invention encompasses a process for evaporating a feedstock comprising at least one polyhydric compound and more than about 0.5% by weight of a salt. The process comprises heating the feedstock to a temperature from about 150° C. to about 300° C. and at a pressure from about 0.01 bars to about 20 bars, wherein at least about 25% by weight of the polyhydric compound in the feedstock evaporates to form a substantially gas phase while the remainder of the polyhydric compound forms a liquid residue stream. The process further comprises cooling at least part of the liquid residue stream to a temperature less than about 120° C. to form a salt precipitate stream. Lastly, the process comprises purging the salt precipitate stream from the process and recycling at least part of the liquid residue stream.

Other aspects and features of the invention will be in part apparent and in part pointed out hereinafter.

### DESCRIPTION OF THE FIGURES

FIG. 1 presents reaction schemes for the conversion of polyhydric compounds having from about four to about twelve carbon atoms to different oxygen-containing products.

FIG. 2 presents a block flow diagram for a process comprising the evaporation of polyhydric compounds having from about four to about twelve carbon atoms and the gas phase reaction of these compounds.

FIG. 3 presents a block flow diagram of a process using flash evaporation and divided-column distillation.

FIG. 4 presents a block flow diagram of a process using guard reactors.

FIG. 5 presents a block flow diagram of a process using the staged introduction of a polyhydric feedstock after each of multiple reaction stages.

FIG. 6 presents a schematic diagram of a dividing wall distillation column used to separate an oxygen-containing product from a reaction product mixture.

FIG. 7 presents a schematic diagram of a process for evaporating a polyhydric feedstock comprising salts and the recycling of the liquid residue stream.

### DETAILED DESCRIPTION

It has been discovered that polyhydric compounds having from about four to about twelve carbon atoms may be evaporated and reacted in gas phase. Preferably, the reactions proceed over a packed bed of a heterogeneous catalyst, which permits ready recovery of the catalyst. Reactions include but are not limited to reactions in the presence of hydrogen, wherein a polyhydric compound having from about four to about twelve carbon atoms is converted to an oxygen-containing product such as acetol, acrolein, glycerol, sorbitol, propylene glycol, furfural, furfural derivatives, and the like.

One aspect of the invention provides a process for converting a polyhydric feedstock comprising at least one polyhydric compound into an oxygen-containing product. The process comprises 1) evaporating the polyhydric feedstock at a temperature between about 150° C. and about 340° C. and at a pressure between about 0.05 bars and about 200 bars to form a substantially gas phase reaction mixture, and 2) contacting the gas phase reaction mixture with a heterogeneous catalyst to facilitate a reaction.

In general, the polyhydric feedstock comprises at least one polyhydric compound and water. The polyhydric compound has from about four to about twelve carbon atoms and more than three hydroxyl groups. In one embodiment, the polyhydric compound may comprise four carbon atoms and more than three hydroxyl groups. In another embodiment, the polyhydric compound may comprise five carbon atoms and more than three hydroxyl groups. In yet another embodiment, the polyhydric compound may comprise six carbon atoms and more than three hydroxyl groups. In still another embodiment, the polyhydric compound may comprise seven carbon atoms and more than three hydroxyl groups. In an alternate embodiment, the polyhydric compound may comprise eight carbon atoms and more than three hydroxyl groups. In another alternate embodiment, the polyhydric compound may comprise nine carbon atoms and more than three hydroxyl groups. In yet another embodiment, the polyhydric compound may comprise ten carbon atoms and more than three hydroxyl groups. In a further embodiment, the polyhydric compound may comprise eleven carbon atoms and more than three hydroxyl groups. In still another one embodiment, the polyhydric compound may comprise twelve carbon atoms and more than three hydroxyl groups. Non-limiting examples of suitable polyhydric compounds include sugars (such as, for example, glucose, fructose, galactose, mannose, sucrose, and the like) and sugar alcohols (such as, for example, sorbitol, mannitol, erythritol, arabitol, maltitol, xylitol, and the like).

In general, the polyhydric compound will have a boiling point higher than about 290° C. The boiling point of the polyhydric compound may be about 295° C., about 300° C., about 305° C., about 310° C., about 315° C., about 320° C., about 325° C., about 330° C., about 335° C., about 340° C., about 345° C., about 350° C., about 355° C., about 360° C., about 365° C., about 370° C., about 375° C., about 380° C., about 385° C., or about 390° C. As an example, the boiling point of sorbitol has been estimated to be about 362° C. (see Example 1).

The polyhydric feedstock typically comprises at least about 2% by weight of the polyhydric compound. In general, the concentration of the polyhydric compound may range from about 2% to about 90% by weight of the feedstock. In one embodiment, the concentration of the polyhydric compound may range from about 2% to about 10% by weight of the feedstock. In another embodiment, the concentration of the polyhydric compound may range from about 10% to about 40% by weight of the feedstock. In a further embodiment, the concentration of the polyhydric compound may range from about 40% to about 90% by weight of the feedstock.

Typically, the polyhydric feedstock also comprises at least about 10% by weight of water. In general, the concentration of water may range from about 10% to about 95% by weight of the feedstock. In one embodiment, the concentration of water may range from about 10% to about 25% by weight of the feedstock. In another embodiment, the concentration of water may range from about 25% to about 50% by weight of the feedstock. In still another embodiment, the concentration of water may range from about 50% to about 95% by weight of the feedstock.

The polyhydric feedstock is generally heated to a temperature from about 150° C. to about 340° C. and a total pressure from about 0.01 bar to about 200 bars to evaporate the feedstock. The polyhydric feedstock may be heated to a temperature ranging from about 150° C. to about 200° C., from about 200° C. to about 250° C., from about 250° C. to about 300° C., or from about 300° C. to about 340° C. In a preferred embodiment, the polyhydric feedstock is heated to a temperature ranging from about 250° C. to about 300° C. The pressure of the evaporating step may range from about 0.01 bar to about 1 bar, from about 1 bar to about 5 bars, from about 5 bars to about 50 bars, or from about 50 bars to about 200 bars. In a preferred embodiment, the pressure may range from about 0.1 bar to about 2 bars. The temperature and the pressure of the evaporating step may be any combination of the above detailed ranges.

Evaporation of the polyhydric feedstock forms a substantially gas phase reaction mixture. In the context of the present invention, the phrase “substantially gas phase” means that there is no liquid in the gas phase reaction mixture. The substantially gas phase reaction mixture may comprise water vapor, however. In general, the concentration of water in the substantially gas phase reaction mixture depends upon the concentration of water in the starting polyhydric feedstock. The concentration of water in the gas phase reaction mixture may range from about 10% to about 95% by weight of the mixture. The concentration of water in the gas phase reaction mixture may range from about 10% to about 25%, from about 25% to about 50%, or from about 50% to about 95% by weight of the mixture.

The process further comprises contacting the substantially gas phase reaction mixture with a heterogenous catalyst to facilitate a reaction such that the oxygen-containing product is formed. Preferably, the heterogeneous catalyst is selected from the group consisting of 1) a catalyst containing at least one element from Groups I or VIII of the Periodic Table, 2) copper, 3) chromium, and 4) an acid catalyst having a Hammett acidity value of less than about +2. In a preferred embodiment, the acid catalyst has a Hammett acidity value from about −2 to about −10.

The reaction may be at least one reaction selected from the group consisting of 1) a dehydration reaction at a temperature between about 180° C. and about 300° C., 2) a hydrocracking reaction at a temperature between about 180° C. and about 300° C., 3) a hydrogenation reaction at a temperature less than about 320° C., and 4) hydrogenolysis reaction at a temperature less than about 320° C. Table 1 summarizes the preferred temperatures for the different reactions.

TABLE 1
Summary of temperature ranges of key reactions.

| | Preferred | | |
|---|---|---|---|
| | Temperature | More Preferred | Most Preferred |
| Reaction | Range | Temperature Range | Temperature Range |
| Dehydration | 180-300° C. | 200-300° C. | 250-300° C. |
| Hydrocracking | 180-300° C. | 200-300° C. | 250-300° C. |
| Hydrogenation | <320° C. | 190-300° C. | 250-300° C. |
| Hydrogenolysis | <320° C. | 190-300° C. | 250-300° C. |

Hydrogenation and hydrogenolysis reactions are preferably performed with gas phase reaction mixtures further comprising water and hydrogen. The ratio of water to polyhydric feedstock may be about 1:0.33, about 1:0.5, about 1:1, about 1:2, or about 1:3. The ratio of hydrogen to polyhydric feedstock may be about 1:0.01, about 1:0.05, about 1:0.25, or about 1:0.5.

Dehydration reactions are also preferably performed with gas phase reaction mixtures further comprising water and hydrogen. The ratio of water to polyhydric feedstock may be about 1:0.33, about 1:0.5, about 1:1, about 1:2, or about 1:3. The ratio of hydrogen to polyhydric feedstock may be about 1:0.5, about 1:1, about 1:2.5, or about 1:5.

Typically, the pressure of the reaction ranges from about 0.01 bar and about 200 bars, and more preferably from about 0.1 bar to about 5 bars. Table 2 summarizes suitable reactions with the preferred pressures of operation. For dehydration reactions, the most preferred pressure is in the range from about 0.1 bar to about 2 bars. For hydrogenation and hydrogenolysis reactions, the most preferred pressure is in the range from about 0.1 bar to about 4 bars.

TABLE 2
Summary of preferred conversions for gas phase reactions over solid
catalyst.

| | Preferred | | Co- | Example | Pressure | Example |
|---|---|---|---|---|---|---|
| Feedstock | Product | Reactions | Reagents | Catalysts | (bar) | Diluents |
| C4-C12 | Propylene | Hydrocracking | Hydrogen | Copper | 0.1-5 | Water |
| polyhydric | Glycol | Dehydration | | Chromite | | Glycerol |
| compound | Acetol | Hydrogenation | | | | |
| | Sorbitol | | | | | |
| Acetol | Acrolein | Dehydration | N/A | Solid Acid | 0.1-5 | Water |
| | | | | Adsorbed | | Hydrogen |
| | | | | Acid | | |
| Glycerol | Acrolein | Dehydration | N/A | Solid Acid | 1-20 | Water |
| | | | | Adsorbed | | Hydrogen |
| | | | | Acid | | |
| C6 & C12 | Sorbitol | Hydrocracking | Hydrogen | Nickel | 0.1-5 | Water |
| polyhydric | | Hydrogenation | | | | |
| compound | | | | | | |
| C4-C12 | Furfural, | Dehydration | N/A | Solid Acid | 0.1-5 | Water |
| polyhydric | Furfural | | | Nickel | | Hydrogen |
| compound | Derivatives | | | | | |

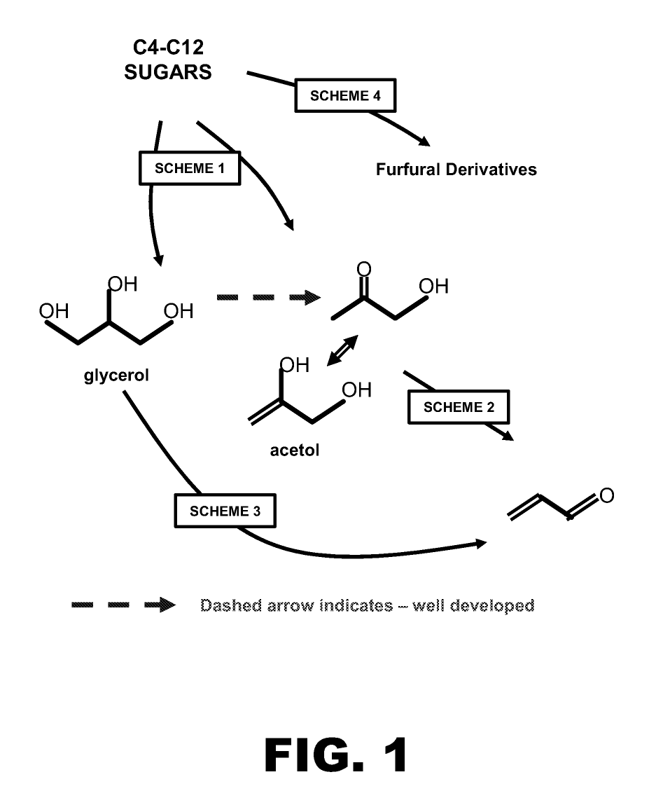

Non-limiting examples of oxygen-containing products formed by the process of the invention include acetol, acrolein, glycerol, sorbitol, propylene glycol, furfural, furfural derivatives, and the like. FIG. 1 summarizes specific reaction schemes for the conversion of polyhydric compounds having from about four to about twelve carbon atoms to various oxygen-containing products. Reaction Scheme 1 refers to the hydrocracking of polyhydric compounds having from about four to about twelve carbon atoms to products having three carbon atoms. Reaction Scheme 2 refers to the dehydration of acetol into acrolein. Reaction Scheme 3 refers to the dehydration of glycerol into acrolein. Reaction Scheme 4 refers to the dehydration of polyhydric compounds having from about four to about twelve carbon atoms into furfural derivatives.

### Gas Phase Reaction of Polyhydric Compounds

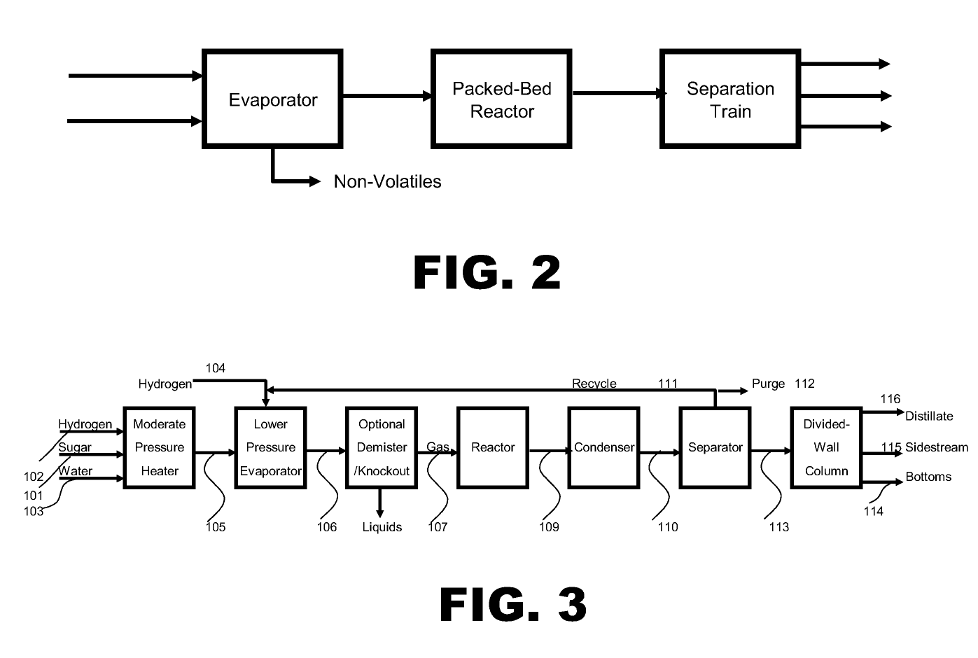

One process of the invention comprises the hydrocracking of a polyhydric compound having from about four to about twelve carbon atoms into a three-carbon compound (i.e., Scheme 1). The general conditions of the process are summarized in Tables 1 and 2. Preferably, the reaction is in a gas phase packed reactor bed comprising the heterogeneous catalyst. FIG. 2 presents a block flow diagram of the preferred process. A polyhydric feedstock comprising at least one polyhydric compound having from about four to about twelve carbon atoms may be mixed with a co-reagent or a diluent such as hydrogen in an evaporator. Non-volatiles such as salts may be purged from the evaporator. The gas phase reaction mixture may enter a reactor for conversion of the feedstock to a product mix. A separation train may condense and separate the products into multiple streams some of which may be recycled in the process.

The hydrocracking process detailed above may produce products such as acetol, propylene glycol, and the like. Generally, the heterogeneous catalysts that are used are those known to be effective for hydrogenation. Non-limiting examples of suitable heterogeneous catalysts include as palladium, nickel, ruthenium, copper, copper zinc, copper chromium, and the like. The polyhydric feedstock generally comprises water from about 10% to about 90% by weight, and more preferably, from about 25% to about 85% by weight of water. The near optimal temperature of the hydrocracking process is about 260° C.

In a preferred embodiment, the process comprises a flash evaporation process and a polyhydric feedstock comprising equal masses of water and polyhydric compound having from about four to about twelve carbon atoms. The polyhydric feedstock along with at least some diluent (which may be water in the feedstock) may be heated to a temperature of about 300° C. in a preheater in the presence of a partial pressure of water of about 3 bars (e.g. a static pressure of 3 bars), after which the pressure may be reduced to about 0.2 bar as it passes through a throttling device such as a control valve to facilitate flash evaporation. After the throttling processing, the feedstock may be mixed with a gas or vapors to facilitate further evaporation. The mixture may be then heated in a heat exchanger to the desired reaction temperature. The gas phase mixture, free of liquids, optionally may be passed through a knockout pot and then introduced to the packed-bed reactor. The gas or vapors mixed with the feedstock after the throttling device are generally at a temperature at least as hot as the feedstock after throttling. The preferred temperature range for the evaporator temperature is from about 250° C. to about 300° C.

Water is effective in reducing by-product formation during gas phase reactions of this invention and generally increases product selectivity. Hydrogen also decreases byproduct formation, thus the preferred gas phase reaction mixtures comprise both hydrogen and water. Without being bound by a particular theory, it is believed that a synergy exists in using the combination of hydrogen and water in the feedstock. In particular, the presence of hydrogen may cause the water to evaporate more evenly over the temperature range from about 70° C. to about 150° C., which may allow for easier heat recovery through counter-current heat exchange between the reactor effluents and the entering reagents. The following preferred conditions with respect to reactor feeds generally apply:

For hydrogenolysis to a three-carbon compound:
Evaporator temperature (maximum reagent temperature in evaporator) between about 180° C. and about 350° C., more preferably between about 250° C. and about 300° C.
Pressure below the dew point pressure and as previously described wherein the gas phase reaction mixture comprises substantially no liquid, has a partial pressure of polyhydric compound between about 0.01 bar and about 0.5 bar of polyhydric compound, more preferably between about 0.02 bar and about 0.1 bar.
Water from 1 part by mass water for every 0.33 to 3 parts polyhydric compound, including the range of about 0.333 to about 1.
Hydrogen at a hydrogen to polyhydric compound molar ratio between about 2:1 and about 100:1 but more preferably between about 3:1 and about 30:1; including a partial pressure of hydrogen between about 0.05 bar and about 2 bars of hydrogen and a total pressure between about 0.1 bars and about 4 bars.
Wherein water and/or hydrogen in the reactor feed allow for the above more-preferred temperatures, for example a temperature of about 275° C. and a water content of about 55% by weight with a hydrogen to polyhydric compound molar ratio of about 6:1 with a finishing reaction to shift equilibrium toward a temperature near 170° C.

In embodiments in which the polyhydric feedstock comprises a six-carbon polyhydric compound, the following reaction conditions and concentrations of the six-carbon polyhydric compound are preferred. At about 0.1 bar of pressure and about 250° C., the preferred concentration of the six-carbon polyhydric compound in water is about 20% by weight. At about 0.1 bar of pressure and about 270° C., the preferred concentration of the six-carbon polyhydric compound in water is about 33% by weight. At about 0.1 bar of pressure and about 290° C., the preferred concentration of the six-carbon polyhydric compound in water is about 50% by weight. In all of these embodiments, the hydrogen to six-carbon polyhydric compound flow rate is from about 8 moles to about 1 mole.

### Further Details on Preferred Evaporation

Exemplary embodiments of this invention encompass the formation of a gas phase mixture comprising hydrogen and the polyhydric compound having from about four to about twelve carbon atoms. Because water is known to reduce by-product formation, its presence is also preferred.

Furthermore, charring may be a problem for polyhydric compounds having four or more carbon atoms. Charring occurs rapidly in the absence of water and at temperatures greater than about 180° C. Thus, the process of the invention reduces charring by manipulating the temperature and pressure of the evaporation step. In one embodiment, a mixture of water and polyhydric compound may be introduced into a moderate pressure heater to attain a temperature from about 210° C. to about 300° C. During this process, heating may cause water to selectively evaporate leading to lower water and higher polyhydric compound concentrations in the liquid phase than is in equilibrium with the vapor phase during the heating. Higher pressures may keep more water in the liquid phase while accumulating higher sensible heats due to higher temperatures.

In a preferred process, the pressure of the polyhydric compound-water mixture may be reduced (e.g. by flow through a valve), and the mixture may be rapidly mixed with a heated vapor or gas (e.g., hydrogen) of similar temperature but lower pressure. The sensible heat and heat in the vapor/gas may allow for rapid evaporation of the polyhydric compound without contact with a heated surface (flash evaporation), wherein the lack of contact with a heated surface substantially eliminates charring.

FIG. 3 illustrates an example of an exemplary process using flash evaporation. Sugar 101, hydrogen 102, and water 103 are heated to a temperature between about 200° C. and about 300° C. in a moderate pressure heater. The mass ratio of sugar 101 and water 103 is preferably between about 0.33 and about 3.0. The pressure in the moderate pressure heater is greater than about 0.9 bars and preferably between about 5 bars and about 30 bars (most preferably about 10 bars). The heated mixture 105 may contain some vapor, but more importantly, the mass ratio of water to sugar in the liquid phase of the mixture is preferably greater than 0.25. The pressure of the heated mixture is reduced to drive evaporation in a lower pressure evaporator. Additional hydrogen 104 is preferably mixed with the heated mixture 105. The hydrogen 104 is preferably at a temperature above about 240° C. when contacted with the heated mixture 105. Heat may be added to the lower pressure evaporator to promote evaporation. The pressure of the lower pressure evaporator is between about 0.01 bar and about 30 bars, but preferably between about 0.1 bar and about 5 bars.

The mostly vaporous mixture 106 exiting the lower pressure evaporator optionally flows through a demisting device to knock out any liquid 108 entrained in the gas/vapor. The gas 107 flows to the reactor. The reactor preferably operates at a pressure slightly less than the lower pressure evaporator. Reactor products 109 flow to a condenser that preferably operates near a temperature of about 40° C. The mixture 110 flows to a separator that separates the gas 111/112 from the liquid 113. The gas is mostly hydrogen and water vapor and may be recycled. The liquid 113 is preferably separated in a divided wall column that produces a distillate containing water 116, a bottoms product 114, and at least one sidestream product 115.

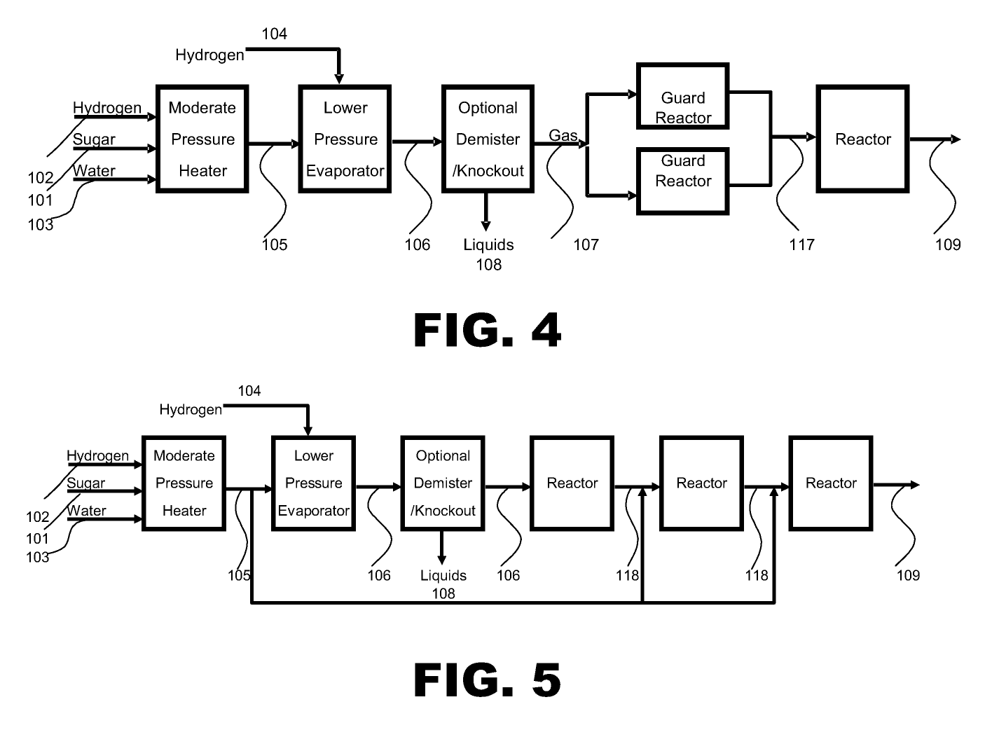

In general, the less volatile the polyhydric compound the greater the tendency for the catalyst to become coated and deactivated. A preferred method to reduce catalyst deactivation is to use guard reactors such as those illustrated by FIG. 4. The gas reaction mixture 107 is directed to one of the guard reactors (valves not shown in block flow diagram) producing a partially reacted mixture 117. This partially reacted mixture 117 is directed to a subsequent reactor to achieve a product 109 with the desired conversion. Guard reactors are in parallel so the catalyst in one may be regenerated while the other allows the overall process to operate without significant disruption. The partially reacted mixture 117 is generally reacted sufficiently to substantially eliminate the tendency for the stream to deactivate the catalyst. The guard reactors optionally operate at a higher temperature, by example, the guard reactors may operate at about 250° C. while the subsequent reactors operate at about 230° C.

An alternative approach to overcoming the deactivation associated with the low volatility of polyhydric compounds may be to operate at lower concentrations of the polyhydric compounds. However, lower concentrations may increase conversion costs. FIG. 5 illustrates a preferred means to overcome problems of higher conversion costs with lower concentrations by incrementally introducing the polyhydric reagents in a series of subsequent reaction sections (which may be in the same or separate reactors). This approach has the added benefit of using the heat of reaction from each pass through a reactor section to assist with evaporation. Flash evaporation is at each point where the product from each stage 118 is mixed with heated mixture of water and reactant 105 from the moderate pressure heater. Preferred operation uses a means to enhance contact between the preheated mixture 105 and the product of each stage 118. Preferred contact means include but are not limited to atomization of the preheated mixture 105 into the product of each stage 118 or use of an inert packing to create an improved surface area for contact and evaporation.

### Divided-Wall Column Distillation

Another aspect of the invention encompasses a separation process for the separation of the oxygen-containing products derived from the polyhydric compounds. Separation costs may be high for oxygen-containing products. These products include but are not limited to propylene glycol, lactic acid, epichlorohydrin, furfual, acetol, and the like. These oxygen-containing products are generated from polyhydric compounds including but not limited to glycerol, sorbitol, sucrose, or glucose. This invention provides an improved method for separating the oxygen-containing products from the reaction product mixture.

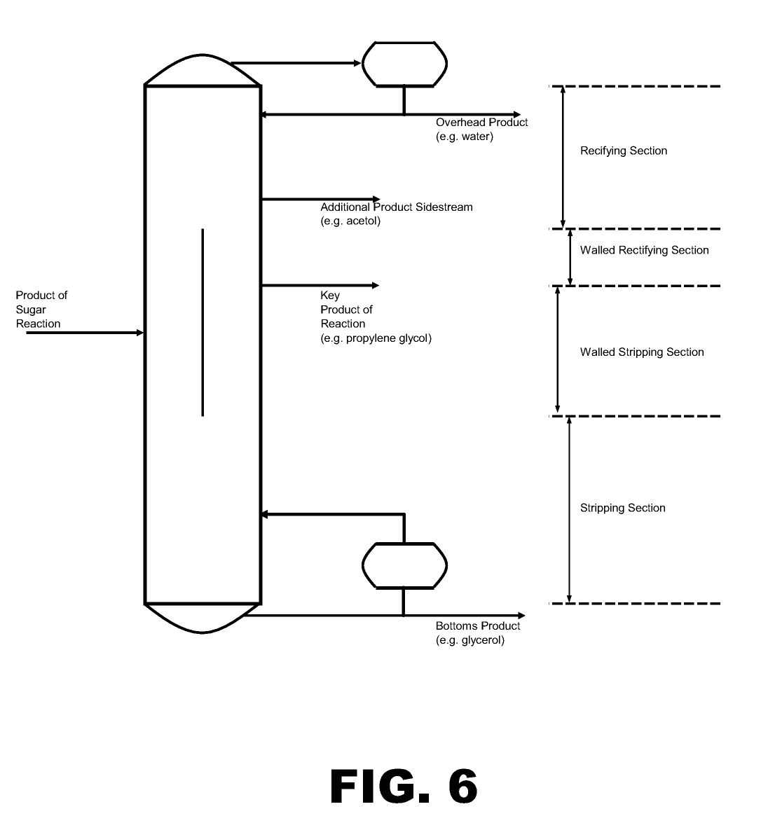

FIG. 6 illustrates an embodiment of the separation process of this invention. The process separates an oxygen-containing product, which may be generated by a reaction process of the invention or via another reaction process, from other compounds of the reaction product mixture. A reaction product mixture, for example, may comprise a mixture of propylene glycol, acetol, water, and glycerol.

The reaction product mixture may be separated in a divided-wall distillation column with four sections as indicated in the diagram (i.e., the column contains rectifying, walled rectifying, walled stripping, and stripping sections). The reaction product mixture is generally introduced into the column at a divided-tray (tray at location in column with dividing wall). The process includes the removal of an oxygen-containing product at a divided tray at the opposite side of the wall relative to the side where the reaction product mixture is fed into the column. The reaction product is not necessarily removed from the column at the same tray as the oxygen-containing product is removed. An example oxygen-containing product is propylene glycol. The column also generally has a bottoms product removed at or below the bottom tray and an overhead product removed at or above the top tray. An additional product or products may be removed as sidestreams at divided trays or trays without dividing walls.

The dividing wall is generally a functionally continuous wall expanding the walled rectifying and walled stripping sections. The wall functionally prevents mixing and vapors in the walled rectifying and walled stripping sections. Each of the four sections (i.e., rectifying, walled rectifying, walled stripping, and stripping sections) of the dividing wall distillation column performs at least about one to about 40 stages of separation and more preferably from about 2 to about 30 stages of separation. Per FIG. 6, the oxygen-containing product tray separates the walled rectifying from the walled stripping sections.

More preferably, the rectifying, walled rectifying, walled stripping, and stripping sections comprise about 4-10 stages, about 4-10 stages, about 8-20 stages, and about 8-20 stages, respectively. In an exemplary embodiment, the rectifying, walled rectifying, walled stripping, and stripping sections comprise about 4 stages, about 4 stages, about 8 stages, and about 8 stages, respectively. The most preferred stages are dependent upon the feedstock composition and the desired degree of separation. In general, the process comprises introducing the reaction product mixture to a dividing wall column, and collecting the oxygen-containing product. In one embodiment, the oxygen-containing product may comprise more than about 10% of water by weight.

Any vapor-liquid separation means may be used to achieve separations (including use of trays and use of packing). High-efficiency packing is generally preferred due to the number of stages of this column. For example, suitable methods include near-stage-located evaporation, near-stage-located condensation, and adjustment of vapor and liquid flow distribution.

### Gas Phase Dehydration of a Three-Carbon Alcohol to Acrolein

Another aspect of the invention encompasses a process for the conversion of a three-carbon alcohol feedstock to acrolein. The three-carbon alcohol may be acetol or glycerol. Tables 1 and 2 summarize exemplary examples of reaction conditions for converting acetol (i.e., Scheme 2) or glycerol (i.e., Scheme 3) to acrolein in a gas phase packed bed reactor. The gas phase conversion of acetol or glycerol to acrolein is demonstrated in Example 7.

The process comprises evaporating the three-carbon alcohol feedstock at a temperature from about 180° C. to about 250° C. and at a pressure from about 0.03 bar to about 5 bars to from a substantially gas phase reaction mixture. The phrase “substantially gas phase” is as defined above. The temperature of the evaporating step may range from about 180° C. to about 200° C., from about 200° C. to about 220° C., or from about 220° C. to about 250° C. The pressure of the evaporating step may range from about 0.03 bar to about 0.2 bar, from about 0.2 bar to about 1 bar, or from about 1 bar to about 5 bars. The temperature and the pressure of the evaporating step may be any combination of the ranges listed above.

The three-carbon alcohol feedstock generally comprises a three-carbon alcohol compound and water. In one embodiment, the three-carbon alcohol compound may be acetol, and the concentration of acetol in water may be about 20%, about 30%, about 40%, about 50%, about 60%, about 70%, about 80%, about 90%, or about 99% by weight. In another embodiment, the three-carbon alcohol compound may be glycerol, and the concentration of glycerol in water may be about 20%, about 30%, about 40%, about 50%, about 60%, about 70%, or about 75% by weight. The three-carbon alcohol feedstock may optionally be combined with other gases (such as, for example, hydrogen).

The process further comprises contacting the substantially gas phase reaction mixture with a heterogeneous catalyst at a temperature that ranges from about 200° C. to about 250° C. The temperature of the reacting step may be from about 200° C. to about 215° C., from about 215° C. to about 230° C., or from about 230° C. to about 250° C. The pressure of the reacting step may range from about 0.03 bar to about 0.2 bar, from about 0.2 bar to about 1 bar, or from about 1 bar to about 5 bars. The temperature and the pressure of the reacting step may be any combination of the ranges detailed above.

The heterogeneous catalyst may be an acidic catalyst having a Hammett acidity value between about −2 and about −10. The acid catalyst may be a solid acid catalyst or a solid, such as activated carbon, with phosphoric acid adsorbed on the catalyst. In a preferred embodiment, the catalyst may be phosphoric acid on an activated carbon. In one embodiment, the acetol feedstock may be a gaseous product of a dehydration reaction of glycerol. In another embodiment, the acetol feedstock may be a liquid comprising at least about 25% by weight of water, and the pressure of the reaction may range from about 0.1 bar to about 0.8 bar (such that lower densities of acrolein are formed at lower water contents). In a further embodiment, the three-carbon alcohol feedstock may be mixed with phosphoric acid prior to the process, wherein upon evaporation of the feedstock, the gas phase reaction mixture comprises less than about 0.5% by weight of phosphoric acid prior to contact with the heterogeneous catalyst. The concentration of phosphoric acid in the vapor phase reaction mixture may be less than about 0.5%, less than about 0.1%, less than about 0.05%, less than about 0.001%, or less than 0.0005% by weight.

Evaporation of Polyhydric Feedstock with Recycling

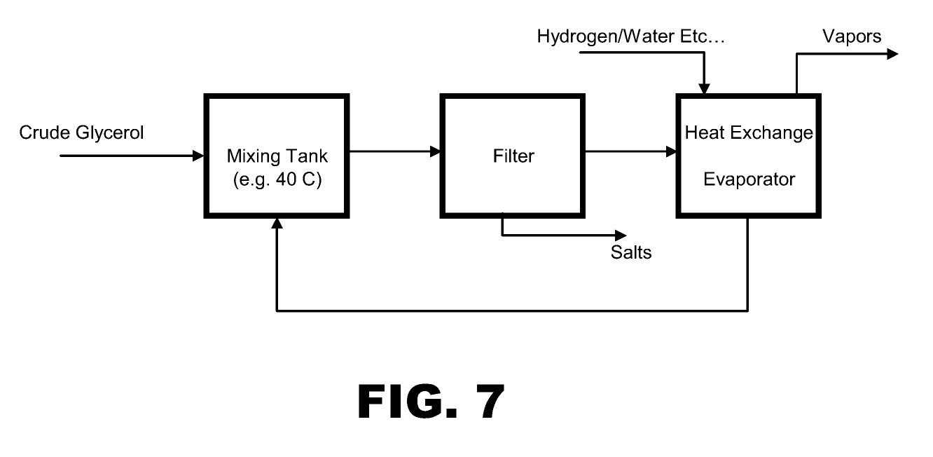

A further aspect of the invention provides a process for evaporating a polyhydric feedstock comprising at least about 0.5% by weight of a salt. The process comprises heating the feedstock to a temperature from about 150° C. to about 300° C. and at a pressure from about 0.01 bar to about 20 bars, wherein at least about 25% by weight of the polyhydric compound in the feedstock evaporates to form a substantially gas phase while the remainder of the polyhydric compound forms a liquid residue stream. The process further comprises cooling at least part of the liquid residue stream to a temperature less than about 120° C. to form a salt precipitate stream. Lastly, the process comprises separating and purging the salt precipitate stream from the process and recycling at least part of the liquid residue stream. FIG. 7 illustrates a preferred embodiment of this process.

In general, the polyhydric feedstock may comprise from about 0.5% to about 10%, or more preferably from about 0.5% to about 5% of a salt. The concentration of salt in the polyhydric feedstock may range from about 0.5% to about 1%, from about 1% to about 2%, from about 2% to about 5%, or from about 5% to about 10% by weight. Non-limiting examples of suitable salts include KCl, NaCl, K₂SO₄, KHSO₄, Na₂SO₄, NaHSO₄, K₃PO₄, K₂HPO₄, KH₂PO₄, Na₃PO₄, Na₂HPO₄, NaH₂PO₄, and combinations thereof. The polyhydric feedstock may comprise at least one polyhydric compound having from about four to about twelve carbon atoms and more than three hydroxyl groups, or the polyhydric feedstock may comprise a three-carbon alcohol. The polyhydric feedstock may further comprise at least about 10% of water by weight.

The evaporating step may comprise heating the polyhydric feedstock comprising the salt to a temperature ranging from about 150° C. to about 200° C., from about 200° C. to about 250° C., or from about 250° C. to about 300° C. The pressure of evaporating step may range from about 0.01 bar to about 0.1 bar, from about 0.1 bar to about 1 bar, from about 1 bar to about 10 bars, or from about 10 bars to about 20 bars. The evaporating step may comprise any combination of the above listed temperature and pressure ranges.

At least a part of the liquid residue steam may be cooled to a temperature less than about 120° C., less than about 100° C., less than about 80° C., less than about 60° C., or less than about 40° C. The salt precipitate stream may be removed from the process using any separation technique known to those of skill in the art. For example, the salt precipitate stream may be separated and purged from the process using a filtering technique. Alternatively, the solid-liquid mixture (i.e., the liquid residue and the salt precipitate) may be passed through a solid-liquid separator (preferably up-flow through a sand bed) that substantially removes the salt precipitate from the liquid residue stream.

The process may further comprise mixing the recycled residue stream with an entering feedstock stream to form a mixture stream. The mixing of the recycled residue stream with an entering feedstock stream essentially cools the recycled residue stream and heats the entering the feedstock stream such that a precipitate is formed. The process further comprises separating the precipitate from the mixture stream, and purging the precipitate from the process.

As various changes could be made in the above-described processes without departing from the scope of the invention, it is intended that all matter contained in the above description and the examples presented below, shall be interpreted as illustrative and not in a limiting sense.

### Examples

The following examples illustrate various embodiments of the invention.

### Example 1

### Vapor Pressure and Evaporation of Sorbitol and Glucose

A thermogravimetric analysis (TGA) method was used to estimate the vapor pressure of sorbitol. Table 3 reports these estimated vapor pressures. An extrapolation of this data estimates the boiling point of sorbitol at 362° C.

TABLE 3
Estimated Vapor Pressure of Sorbitol.

| | Estimated vapor |
|---|---|
| Temperature (° C.) | pressure (bar) |
| 250 | 0.013 |
| 275 | 0.045 |
| 300 | 0.123 |
| 325 | 0.30 |
| 350 | 0.65 |

An equal-mass mixture of sorbitol (MW=182) and water (MW=18) would have a sorbitol mole fraction of 9 mol %. Based on Raoult's Law, the dew point pressure of this 50:50 mixture at 250° C. and 275° C. are about 0.14 bars and 0.5 bars, respectively. Addition of hydrogen to the mixture would further increase the dew point.

The evaporation and subsequent condensation of sorbitol was evaluated in a continuous flow process with feed at 2.5 wt %. The data are summarized in Table 4.

TABLE 4
The Effect of Temperature on Sorbitol Evaporation.

| Temperature | Pressure | Recovery of Non- | Recovery of |
|---|---|---|---|
| (° C.) | (bar) | Volatiles¹ (%) | Sorbitol² (%) |
| 280 | 0.1 | 52 | 51 |
| 285 | 0.1 | 65 | 63 |
| 290 | 0.1 | 78 | 76 |
| 292 | 0.1 | 84 | 82 |
| 294 | 0.1 | 91 | 89 |

¹Recovery calculated from TGA analysis.
²Recovery calculated from HPLC analysis.

The condensed aqueous-sorbitol mixture was clear, without odor, and absent of degradation products. The data of Table 4 validate the hypothesis that sorbitol can be successfully evaporated to a gas phase and recovered as a condensate.

The evaporation and condensation of sorbitol 4 had recoveries less than would be expected based on the TGA estimates of vapor pressure. Higher water content, hydrogen dilution, and flash evaporation methods could be used promote sorbitol evaporation in preferred processes. Based on available data and extrapolating to account for improved flash evaporation means (as compared to the experimental system of Table 4), the more preferred operating conditions for a 50 wt % solution of sorbitol in water are about 0.01 to 0.04 bars at 250° C. and 0.04-0.1 bars at 275° C. The pressure range is under the dew point pressure, preferably with a margin of safety.

Evaporation of glucose at similar condition resulted is substantial degradation of the glucose. Enhanced flash evaporation methods are needed that would minimize the time at which glucose is on hot surfaces to promote evaporation.

### Example 2

### Vapor Pressure and Evaporation of Sorbitol

The gas phase conversions of glycerol, sorbitol, and glucose were carried out at the temperature range from 240° C. to 290° C. and a pressure of 1 bar. The reaction system consisted of evaporator, trap, fix-bed reactor and ice-water condenser. For each run, 70 g of catalyst was loaded into copper fix-bed reactor (i.d.=1 inch). While the system reached the desired temperature and pressure, the aqueous sugar/sugar alcohol solution (mass concentration in solution was 2.5%) was pumped into reaction system by a micro-pump at the flow rate of approximately 200 gram per hour. Hydrogen was fed at 7-8 l/min. The samples were collected from ice-water condenser and analyzed by Hewlett-Packard gas chromatography (GC) and high performance liquid chromatography (HPLC) without further delay. Table 5 summarizes the conversions of the two prominent products for these reactions.

TABLE 5
Gas Phase Reaction Conversions of Glycerol, Sorbitol, and Glucose . . .

| Substrate | Catalyst* | Temp (° C.) | Prod. 1 | Yield (%) | Product 2 | Yield (%) |
|---|---|---|---|---|---|---|
| glycerol | Copper | 200 | acetol | 16 | propylene | 24 |
| | | 220 | | 32 | glycol | 43 |
| | | 240 | | 27 | | 22 |
| | | 260 | | 12 | | 6.0 |
| glycerol | Palladium | 200 | acetol | 1.7 | 1-propanol | 0.55 |
| | | 220 | | 9 | | 2.9 |
| | | 240 | | 11 | | 1.8 |
| | | 260 | | 4.7 | | 1.1 |
| sorbitol | Copper | 240 | acetol | 1.9 | 3-hydroxy- | 2.4 |
| | | 260 | | 6.1 | 2-butanone | 7.8 |
| | | 280 | | 12 | | 3.7 |
| sorbitol | Palladium | 240 | acetol | 0.57 | 3-hydroxy- | 1.0 |
| | | 260 | | 1.4 | 2-butanone | 1.5 |
| | | 280 | | 9.1 | | 15 |
| glucose | Copper | 240 | acetol | 2.5 | 3- | 5.7 |
| | | 260 | | 6.8 | furaldehyde | 15 |
| | | 280 | | 9.0 | | 8.9 |
| glucose | Palladium | 240 | acetol | 0.88 | methoxyfurfural | 5.3 |
| | | 260 | | 1.01 | | 13 |
| | | 280 | | 1.13 | | 55 |

*Copper was copper-chromite catalyst and palladium was ~1% palladium on carbon.

The results of Table 5 validate the gas phase reactions of C4-C12 sugar embodiments of this invention. The trap prior to the reactor assured that only gases made it through the reactor. The changing conversions with different catalysts indicated that the conversions were catalyst-dependent (not homogeneous). The product yields were calculated based on a normalization method. A GCMS library was used to identify the products (other than acetol and propylene glycol) with said products being the most probable but with less than 100% certainty in the identification.

### Example 3

### Preliminary Data on the Hydrogenation of Sorbitol

Preliminary data were obtained for the hydrogenation of sorbitol in the gas phase over a solid catalyst. Prior to operating the reactor, the evaporation was evaluated at 80% water and an excess of hydrogen at temperatures of 260° C. and 290° C. A trap between the evaporator and condenser was estimated to knock out about a fifth of the sorbitol, indicating that about four-fifths had evaporated—this evaporation was performed for about 3 hours. Next, the plug flow reactor was placed between the evaporator and condenser.

The GC registered >90% selectivity to the mixture of acetol and propylene glycol (known to be in equilibrium). Variations in the reactor temperature produced different ratios of acetol and propylene glycol that are completely consistent with trends observed with the hydrogenolysis of glycerol.

Ethylene glycol is formed from the direct hydrogenolysis of glycerol. It appears that the essential absence of glycerol in the sorbitol reaction (it immediately reacts in gas phase) leads to essentially zero ethylene glycol production—actually an improvement over the glycerol reaction.

These preliminary runs were operated at about 1.2 bars of pressure. Pressure is an important degree of freedom—lower pressures can result in the need for lower water contents (e.g. 25% versus 80%) and evaporation at lower temperatures (totally avoiding forming of oligomers). It is anticipated that fructose and glucose will exhibit similar volatility and reactivity as sorbitol.

The hypothesized mechanism includes the following steps: 1) adsorption of C6 sugar onto catalyst; 2) hydrocracking of C6 sugar to glycerol and another C3 compound; 3) essentially immediate dehydration of glycerol to acetol, and 4) rapidly established chemical equilibrium between acetol and propylene glycol.

### Example 4

### Base Case Glycerol Conversion with Copper Chromite

The gas-phase reaction of glycerol was performed in the reactor systems illustrated by FIGS. 2 and 6. Performance is summarized in Table 6. The data establish base-case performance for the adiabatic reactor and isothermal reactor, respectively. The lower amount of “other” products of the isothermal reactor indicates superior performance since these other products were a mix of components present in low concentrations—many of which are of little value. The believed reason for poorer performance in the adiabatic reactor is the exothermic reaction increases reactor temperature that favors acetol in the acetol-propylene glycol equilibrium. The acetol is more reactive to these other products.

The isothermal data illustrates that good conversions and selectivities were attained with the gas phase reaction at the specified conditions. The impact of water in going from 60% to 90% water was minimal. When the heat of reaction was allowed to increase the reaction temperature, less propylene glycol was formed and more of the product remained as acetol (equilibrium limitations). Acetol tends to be more reactive and leads to more undesirable by-products.

### Example 5

### Sorbitol Conversion with Copper Chromite

The gas-phase reaction of sorbitol was performed in the reactor systems illustrated by FIGS. 2 and 6. Performance is summarized in Table 7. The data establishes that sorbitol has reaction trends similar to glycerol. Good performance was attained. Sorbitol is less volatile than glycerol, and so, higher water contents are required to facilitate evaporation. At these higher water contents, the results tend to be similar to the glycerol conversion. An explanation for these results (being similar to glycerol conversion) is that the gas phase reaction of sorbitol at these conditions leads to an initial glycerol intermediate that reacts in a manner similar to glycerol.

### Example 6

### Glucose Conversion with Copper Chromite

The gas-phase reaction of sorbitol was performed in the reactor systems illustrated by FIGS. 2 and 6. Performance is summarized in Table 8. The data establishes that glucose has good conversions similar to sorbitol and glycerol; however, high amounts of furfural were formed. Higher hydrogen partial pressures will favor formation of products other than furfural. More-isothermal performance will favor formation of propylene glycol. These data indicate that this process is effective for production of methoxy furfural.

TABLE 6
Reaction Conditions and Conversion Data for Glycerol in Gas-Phase Reactor.

| | | | | | | | Feed | | | | | | |
|---|---|---|---|---|---|---|---|---|---|---|---|---|---|
| | | Catalyst | Hydrogen | Percent | | | Flow | Sugar | | Propy. | | | |
| | Reactor | Loading | Flow Rate | Water | | Reactor | Rate | Conversion | Acetol | Glycol | | | |
| Run | Type* | (g) | (l/min) | in Feed | Evap. T | Entrance T | (g/h) | (%) | (%) | (%) | Prod. 3 | Prod. 4 | Other |
| 6 | A-cool | 25 | 7.5 | 80 | 235 | 230 | 210 | 98 | 68.8 | 0.0 | 4.7 | 7.5 | 19.1 |
| 7 | A-cool | 25 | 3.5 | 80 | 235 | 229 | 210 | 95 | 71.0 | 0.0 | 1.3 | 3.4 | 24.4 |
| 93 | A-cool | 25 | 4.5 | 60 | 240 | 232 | 215 | 98 | 45.0 | 7.7 | 12.5 | 0.0 | 35.0 |
| 94 | A-cool | 25 | 5.5 | 60 | 240 | 229 | 215 | 99 | 68.0 | 0.0 | 4.2 | 0.0 | 27.9 |
| 95 | A-cool | 25 | 7.5 | 60 | 240 | 226 | 215 | 99 | 37.4 | 0.0 | 12.0 | 6.0 | 44.6 |
| 105 | A-cool | 25 | 3.5 | 65 | 235 | 230 | 210 | 99 | 37.8 | 0.0 | 62.2 | 0.0 | 0.0 |
| 106 | A-cool | 25 | 6.5 | 65 | 235 | 228 | 210 | 99 | 48.6 | 0.0 | 51.4 | 0.0 | 0.0 |
| 108 | A-cool | 25 | 3.5 | 65 | 235 | 231 | 210 | 99 | 76.1 | 0.0 | 23.8 | 0.0 | 0.1 |
| 124 | cobra | 193 | 6.5 | 80 | 235 | 231 | 210 | 100 | 45.2 | 41.0 | 3.8 | 1.0 | 9.0 |
| 125 | cobra | 193 | 6.5 | 80 | 235 | 229 | 210 | 100 | 45.4 | 44.4 | 2.9 | 2.1 | 5.3 |
| 126 | cobra | 193 | 5.5 | 60 | 240 | 231 | 215 | 80 | 46.7 | 40.9 | 2.9 | 2.9 | 6.7 |
| 127 | cobra | 193 | 5.5 | 60 | 240 | 229 | 215 | 80 | 41.6 | 43.5 | 4.3 | 2.9 | 7.7 |
| 128 | cobra | 193 | 4.5 | 60 | 240 | 232 | 215 | 75 | 52.6 | 28.6 | 2.7 | 2.5 | 13.6 |
| 129 | cobra | 193 | 6.5 | 60 | 240 | 228 | 215 | 100 | 53.1 | 36.9 | 3.2 | 1.3 | 5.6 |
| 142 | cobra | 193 | 5.5 | 70 | 235 | 231 | 215 | 97 | 42.8 | 50.6 | 2.7 | 1.9 | 2.1 |
| 143 | cobra | 193 | 4.5 | 70 | 235 | 229 | 215 | 95 | 40.4 | 48.5 | 4.7 | 3.5 | 2.9 |
| 144 | cobra | 193 | 6.5 | 70 | 235 | 227 | 215 | 97 | 44.5 | 48.8 | 3.0 | 1.7 | 2.0 |
| 145 | cobra | 193 | 6.5 | 60 | 240 | 231 | 215 | 95 | 47.2 | 45.1 | 3.7 | 1.9 | 2.2 |
| 146 | cobra | 193 | 6.5 | 60 | 240 | 230 | 215 | 94 | 41.6 | 51.0 | 3.2 | 1.6 | 2.6 |
| 147 | cobra | 193 | 5.5 | 60 | 240 | 231 | 215 | 94 | 51.4 | 41.8 | 3.1 | 1.6 | 2.2 |

*A-cool = air-cooled

TABLE 7
Reaction Conditions and Conversion Data for Sorbitol in Gas-Phase Reactor

| | | | Hydrogen | | | | Feed | | | | | | |
|---|---|---|---|---|---|---|---|---|---|---|---|---|---|
| | | Catalyst | Flow | Percent | | | Flow | Sugar | | Propy. | | | |
| | Reactor | Loading | Rate | Water | | Reactor | Rate | Conversion | Acetol | Glycol | | | |
| Run | Type* | (g) | (l/min) | in Feed | Evap. T | Entrance T | (g/h) | (%) | (%) | (%) | Prod 3 | Prod 4 | Other |
| 9 | A-cool | 25 | 3.5 | 95 | 235 | 230 | 210 | 95 | 82.1 | 0.0 | 3.4 | 1.3 | 13.2 |
| 15 | A-cool | 25 | 5.5 | 92.5 | 240 | 233 | 195 | 94 | 98.0 | 0.0 | 0.0 | 0.0 | 2.0 |
| 16 | A-cool | 25 | 3.5 | 92.5 | 240 | 230 | 195 | 88 | 83.0 | 0.0 | 17.0 | 0.0 | 0.0 |
| 25 | A-cool | 25 | 3.5 | 90 | 240 | 235 | 220 | 85 | 47.1 | 0.0 | 23.2 | 29.2 | 0.5 |
| 27 | A-cool | 25 | 3.5 | 90 | 240 | 233 | 220 | 85 | 84.1 | 0.0 | 15.9 | 0.0 | 0.0 |
| 59 | A-cool | 25 | 4.5 | 94 | 245 | 231 | 198 | 97 | 16.8 | 9.5 | 13.4 | 12.2 | 48.0 |
| 66 | A-cool | 25 | 4.5 | 94 | 240 | 229 | 198 | 95 | 16.2 | 9.8 | 15.4 | 11.9 | 46.8 |
| 83 | A-cool | 25 | 6.5 | 85 | 240 | 231 | 185 | 80 | | | | | |
| 84 | A-cool | 25 | 6.5 | 85 | 245 | 230 | 185 | 78 | | | | | |
| 113 | A-cool | 25 | 5.5 | 75 | 240 | 229 | 194 | 85 | 66.9 | 0.0 | 15.4 | 2.3 | 15.4 |
| 114 | A-cool | 25 | 6.5 | 75 | 240 | 231 | 194 | 85 | 67.2 | 0.0 | 25.1 | 7.7 | 0.0 |
| 116 | A-cool | 25 | 4.5 | 75 | 240 | 229 | 194 | 80 | 56.0 | 0.0 | 20.7 | 7.3 | 16.0 |
| 148 | cobra | 193 | 5.5 | 90 | 240 | 233 | 215 | 98 | 47.4 | 47.4 | 2.8 | 1.0 | 1.4 |
| 149 | cobra | 193 | 5.5 | 90 | 241 | 232 | 215 | 99 | 46.5 | 46.7 | 4.2 | 1.5 | 1.1 |
| 150 | cobra | 193 | 5.5 | 90 | 240 | 233 | 215 | 99 | 41.3 | 48.5 | 5.4 | 1.4 | 3.5 |

*A-cool = air-cooled

TABLE 8
Reaction Conditions and Conversion Data for Glucose in Gas-Phase Reactor.

| | | | Hydrogen | | | | Feed | | | | | |
|---|---|---|---|---|---|---|---|---|---|---|---|---|
| | | Catalyst | Flow | Percent | | | Flow | Sugar | | Propy. | | |
| | Reactor | Loading | Rate | Water | | Reactor | Rate | Conversion | Acetol | Glycol | | |
| Run | Type* | (g) | (l/min) | in Feed | Evap. T | Entrance T | (g/h) | (%) | (%) | (%) | Product 3 | Product 4 |
| 33 | A-cool | 25 | 4.5 | 95 | 240 | 232 | 200 | 97 | 26.2 | 0.0 | 22.6 | 8.9 |
| 34 | A-cool | 25 | 5.5 | 95 | 242 | 231 | 200 | 98 | 24.1 | 0.0 | 22.2 | 19.3 |

*A-cool = air-cooled

### Example 7

### Dehydration of Acetol to Acrolein

Catalyst was prepared by taking 30 grams of activated carbon and soaking the carbon in about 60 grams of a 40% by weight phosphoric acid solution. After an hour at 25° C., free liquid was drained and the solids were dried in an oven at about 100° C. The catalyst was loaded in a 0.5″ ID tube to form a packed be reactor.

Feed mixtures prepared for separate reactor runs included 20% glycerol in water and 50% acetol in water. Feed mixtures were fed through a heat exchanger at about 200 ml/hr to a temperature of 290° C. to form a vapor phase that was sent through a knockout drum to remove entrained liquids. The liquid-free mixture was then fed to the packed-bed reactor maintained at 250° C. The reactor effluents were condensed to a temperature of about 40° C. and the liquid product was evaluated using gas chromatography. Table 9 summarizes the compositions for the two feed compositions.

TABLE 9
Preliminary Data on Conversion of 3-carbon Alcohols to Acrolein.

| | | Acrolein | |
|---|---|---|---|
| Feed | Conversion | Selectivity | Comments on Sample |
| Glycerol | 85% | >55% | Pungent smell of acrolein |
| Acetol | 100% | >55% | Pungent smell of acrolein |

Preliminary data in Table 9 confirms that acetol does undergo this reaction with higher conversions than when glycerol is the feed. A process option would be to feed gas phase products from a glycerol-to-acetol conversion over a copper-chromite catalyst directly into a gas phase reactor with solid acid catalyst to produce an overall acrolein yield in excess of 85%. Higher yields would be expected at lower temperatures.

What is claimed is:

1. A process for converting a polyhydric feedstock into an oxygen-containing product, the polyhydric feedstock comprising at least about 10% by weight of water and at least about 2% by weight of at least one polyhydric compound, the polyhydric compound having from about four to about twelve carbon atoms and more than three hydroxyl groups, the process comprising:
a. evaporating the feedstock at a temperature from about 150° C. to about 340° C. and at a pressure from about 0.01 bar to about 200 bars to form a substantially gas phase reaction mixture; and
b. contacting the gas phase reaction mixture with a heterogeneous catalyst to facilitate a reaction such that the oxygen-containing product is formed.

2. The process of claim 1, wherein the temperature is from about 250° C. to about 300° C. and the pressure is from about 0.1 bar to about 2 bars.

3. The process of claim 1, wherein the polyhydric compound is selected from the group consisting of a sugar and a sugar alcohol.

4. The process of claim 3, wherein the reaction is at least one reaction selected from the group consisting of a dehydration reaction occurring at a temperature from about 180° C. to about 300° C., a hydrocracking reaction occurring at a temperature from about 180° C. to about 300° C., a hydrogenation reaction occurring at a temperature less than about 320° C., and a hydrogenolysis reaction occurring at a temperature less than about 320° C.

5. The process of claim 4, wherein the heterogeneous catalyst is at least one catalyst selected from the group consisting of a catalyst comprising at least one element from Groups I or VIII of the Periodic Table and an acid catalyst having a Hammett acidity value of less than about +2.

6. The process of claim 5, wherein the acid catalyst has a Hammett acidity value from about −2 to about −10.

7. The process of claim 5, wherein the reaction is a hydrogenolysis reaction, and the gas phase reaction mixture further comprises water and hydrogen, the ratio of water to polyhydric feedstock is from about 1:0.33 to about 1:3, and the ratio of hydrogen to polyhydric feedstock is from about 1:0.01 to about 1:0.5.

8. The process of claim 5, wherein the reaction is dehydration, and the gas phase reaction mixture further comprises water and hydrogen, the ratio of water to polyhydric feedstock is from about 1:0.33 to about 1:3, and the ratio of hydrogen to polyhydric feedstock is from about 1:0.5 to about 1:5.

9. The process of claim 5, wherein the oxygen-containing product is selected from the group consisting of acetol, acrolein, glycerol, sorbitol, propylene glycol, furfural, and a furfural derivative.

10. The process of claim 5, wherein the temperature is from about 250° C. to about 300° C. and the pressure is from about 0.1 bar to about 2 bars.

11. The process of claim 1, wherein the gas phase reaction mixture further comprises hydrogen, and the process further comprises separating the oxygen-containing product from the reaction mixture in a dividing wall column comprising a rectifying section comprised of more than four stages, a walled rectifying section comprised of more than four stages, a walled stripping section comprised of more than eight stages, and a stripping section comprised of more than eight stages.

12. The process of claim 1, wherein the feedstock further comprises more than about 0.5% by weight of a salt, the feedstock being heated to a temperature from about 150° C. to about 300° C. and at a pressure from about 0.02 bar to about 20 bars such that at least about 25% by weight of the polyhydric compound in the feed stock is evaporated to form the gas phase reaction mixture while the remainder of the polyhydric compound forms a liquid residue stream, the process further comprising:
a. cooling at least part of the liquid residue stream to a temperature less than about 120° C. to form a salt precipitate stream;
b. purging the precipitate stream from the process; and
c. recycling at least part of the liquid residue stream.

13. The process of claim 12, further comprising:
a. mixing the recycled residue stream with an entering feedstock stream to form a mixture stream, whereby the residue stream is cooled and the feedstock stream is heated such that a precipitate is formed;
b. separating the precipitate from the mixture stream; and
c. purging the precipitate from the process.

14. A process for separating an oxygen-containing product from a reaction product mixture comprising a polyhydric compound and hydrogen, the process comprising:
a. introducing the reaction product mixture into a dividing wall column comprised of a rectifying section comprised of more than four stages, a walled rectifying section comprised of more than four stages, a walled stripping section comprised of more than eight stages, and a stripping section comprised of more than eight stages; and
b. collecting the oxygen-containing product.

15. The process of claim 14, wherein the product comprises more than about 10% by weight of water.

16. A process for converting a three-carbon alcohol feedstock to acrolein, the process comprising:
a. evaporating the alcohol feedstock at a temperature from about 180° C. to about 250° C. and at a pressure from about 0.03 bar to about 5 bars to form a substantially gas phase reaction mixture; and
b. contacting the reaction mixture with a heterogeneous catalyst at a temperature from about 200° C. to about 250° C., the catalyst having a Hammett acidity value from about −2 to about −10.

17. The process of claim 16, wherein the three-carbon alcohol is selected from the group consisting of acetol and glycerol.

18. The process of claim 16, wherein the feedstock further comprises at least about 25% by weight of water, and the pressure is from about 0.1 bar to about 0.8 bar.

19. The process of claim 16, wherein the catalyst is phosphoric acid on an activated carbon.

20. The process of claim 16, wherein phosphoric acid is mixed with the feedstock prior to the process, and the reaction mixture comprises less than about 0.5% by weight of phosphoric acid prior to contact with the catalyst.

21. A process for evaporating a feedstock comprising at least one polyhydric compound and more than about 0.5% by weight of a salt, the process comprising:
a. heating the feedstock to a temperature from about 150° C. to about 300° C. and at a pressure from about 0.01 bar to about 20 bars, wherein at least about 25% by weight of the polyhydric compound in the feedstock evaporates to form a substantially gas phase while the remainder of the polyhydric compound forms a liquid residue stream;
b. cooling at least part of the liquid residue stream to a temperature less than about 120° C. to form a salt precipitate stream;
c. purging the salt precipitate stream from the process; and
d. recycling at least part of the liquid residue stream.

22. The process of claim 21, further comprising:
a. mixing the recycled residue stream with an entering feedstock stream to form a mixture stream, whereby the residue stream is cooled and the feedstock stream is heated such that a precipitate is formed;
b. separating the precipitate from the mixture stream; and
c. purging the precipitate from the process.
